(19) United States
(12) Patent Application Publication
(10) Pub. No.: US 2010/0151044 A1
(43) Pub. Date: Jun. 17, 2010

(54) USE OF ISOOSMOTIC SEAWATER-BASED IONIC SOLUTIONS FOR MANUFACTURING MEDICAL DEVICES FOR THE PREVENTION OF COMPLICATIONS OF THE COMMON COLD OR OF THE FLU SYNDROME

(75) Inventors: Anne Beaulieu, Dinard (FR); Francois Andro, Hede (FR)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: LABORATOIRE DE LA MER, Saint Malo (FR)

(21) Appl. No.: 12/596,955

(22) PCT Filed: Apr. 25, 2008
(86) PCT No.: PCT/FR08/50760

§ 371 Date: Oct. 21, 2009

(30) Foreign Application Priority Data
Apr. 26, 2007 (FR) 0703042

(51) Int. Cl. A61K 33/34; A61P 31/16; A61K 33/20; A61K 33/26; A61K 33/30; A61K 33/32

(52) U.S. Cl. 424/630; 424/663; 424/646; 424/641; 424/639

(57) ABSTRACT

A method of using a seawater-based isoosmotic ionic solution for manufacturing a medical device for administering said solution by nasal spray or mist to patients in the remission phase of the common cold or flu syndrome, or to patients suffering from the common cold or flu syndrome, to prevent and/or treat complications of the common cold or flu syndrome is described.

### CROSS-REFERENCE TO RELATED APPLICATIONS

This application claims the benefit of International Patent Application No. PCT/FR2008/050760, filed on Apr. 25, 2008, which claims priority to French Patent Application No. 0703042, filed Apr. 26, 2007, both of which applications are hereby incorporated by reference in their entirety.

### BACKGROUND OF THE INVENTION

1. Field of the Invention

The invention relates to the use of seawater-based isoosmotic solutions for preparing medical devices for the prevention of the complications of the common cold or flu syndrome.

2. Background of the Invention

The Applicant has already described the application of seawater-based isoosmotic ionic solutions for preventing and limiting the release of the chemical mediators responsible for triggering inflammatory phenomena of the bronchial and pulmonary mucosa, particularly in the patent EP1091747, but also for a cerumenolytic treatment, in the patent EP1091746.

Furthermore, the Applicant has developed water-based ionic solutions of the type in question, enriched with potassium for use for treating and washing eyes and for use as a contact lens rinse product. These solutions and the uses thereof are particularly described in the patents FR2843029 and FR2803205.

### SUMMARY OF THE INVENTION

The invention provides a method of using a seawater-based isoosmotic ionic solution for manufacturing a medical device for administering said solution by nasal spray or mist to patients in the remission phase of the common cold or flu syndrome, or to patients suffering from the common cold or flu syndrome, to prevent and/or treat complications of the common cold or flu syndrome. In one embodiment, the seawater-based isoosmotic ionic solution has:
a pH of 7.8 to 8.4,
a dry matter content of 1 to 2% by weight,
an osmolarity of 250 to 350 mOsm/kg, preferentially 305 to 315 mOsm/kg, and the following main constituent contents:
500 to 2600 mg/l of sodium (Na),
40 to 6500 mg/l of potassium (K),
5800 to 6000 mg/l of chloride (Cl),
20 to 400 mg/l of calcium (Ca),
50 to 1500 mg/l of magnesium (Mg).

### BRIEF DESCRIPTION OF THE FIGURES

FIG. 1 is a graphic representation of the results shown in Table 1.

FIG. 2 is a graphic representation of the results shown in Table 2.

FIG. 3 is a graphic representation of the results shown in Table 3.

FIG. 4 is a graphic representation of the results shown in Table 4.

FIG. 5 is a graphic representation of the results shown in Table 5.

FIG. 6 is a graphic representation of the results shown in Table 6.

FIG. 7 is a graphic representation of the results shown in Table 7.

FIG. 8 is a graphic representation of the results shown in Table 8.

FIG. 9 is a graphic representation of the results shown in Table 9.

FIG. 10 is a graphic representation of the results shown in Table 10.

FIG. 11 is a graphic representation of the results shown in Table 11.

FIG. 12 is a graphic representation of the results shown in Table 12.

FIG. 13 is a graphic representation of the results shown in Table 13.

### DETAILED DESCRIPTION

In the present invention, the term seawater-based isoosmotic ionic solution or composition refers to any seawater-based solution, i.e. containing more than 30% by weight of seawater, preferentially more than 75% by weight of seawater, which has an osmolarity of 250 to 350 mOsm/kg, preferentially from 305 to 315 mOsm/kg. This definition does not cover so-called physiological solutions which only contain ionic species such as Na and Cl and, optionally, Se ions.

The common cold is the most frequent infectious, contagious and viral disease encountered by humans. On average, adults suffer from the common cold two to four times a year and children can experience up to twelve episodes of the common cold a year. The common cold is accompanied by nasal symptoms such as stinging in the nasal cavity, sneezing, runny nose of varying severity and a blocked nose sensation, but also sore throat, fever and general malaise which may result in absenteeism. The common cold is a rhinovirus disease, subject to spontaneous remission after approximately one week with residual coughing liable to last for up to three weeks. Patients suffering from flu syndrome display similar symptoms even though the family of viruses involved is not the same.

In the present invention, the term patients suffering from the common cold or flu syndrome refers to patients displaying the main symptoms observed during the first week of the cold and the term patients in the remission phase refers to those displaying residual coughing.

In view of the general condition of the patient during these episodes, the patient is less resistant to bacterial attacks. In this way, patients in the remission phase of the common cold or flu syndrome or patients suffering from the common cold or flu syndrome frequently display complications. In the present invention, the term complication of the common cold or flu syndrome refers to bacterial throat, bronchial, rhinopharyngeal, ear and sinus infections. These complications may require the use of antibiotics. Furthermore, the treatment of the common cold or flu syndrome is frequently accompanied by the administration of antiinflammatories, antiinfectives, antipyretics, mucolytics, nasal decongestants and cough medicines.

However, in the long term, such treatments cause extensive side effects and/or patients no longer tolerate these treatments.

Therefore, there is a genuine need for a well-tolerated product for long-term treatments, which does not cause any side effects and makes it possible to prevent any complication of the common cold or flu syndrome, while reducing or eliminating the administration of conventional additional medicinal products such as, in particularly, antiinflammatories, antipyretics, antiinfectives, nasal decongestants and cough medicines.

The Applicant unexpectedly and surprisingly discovered that the administration, by means of a daily nasal spray or mist, of a seawater-based isoosmotic ionic solution during and after an episode of the common cold or flu syndrome, makes it possible to prevent complications, avoid relapses, reduce the number and frequency of episodes of the common cold and improve the general state of health of patients more rapidly while avoiding the administration of medicinal products.

In this way, the present invention relates to the use of a seawater-based isoosmotic ionic solution for manufacturing a medical device for administering said solution by nasal spray or mist to patients in the remission phase of the common cold or flu syndrome, or to patients suffering from the common cold or flu syndrome, to prevent and/or treat complications of the common cold or flu syndrome.

According to the present invention, the medical device is a container provided with a nasal spray or mist tube containing said seawater-based isoosmotic solution.

The invention is intended both for the treatment of adult patients and children or infants. According to one advantageous embodiment, when the patient is an adult, the solution is applied by means of a nasal spray. When the patient is a child or an infant, nasal mist application is preferred.

In the present application, the number of daily sprays or mists mentioned is given for each nasal cavity.

More specifically, the seawater-based isoosmotic ionic solution has:
a pH of 7.8 to 8.4,
a dry matter content of 1 to 2% by weight,
an osmolarity of 250 to 350 mOsm/kg, preferentially 305 to 315 mOsm/kg, and the following main constituent contents:
500 to 2600 mg/l of sodium (Na),
40 to 6500 mg/l of potassium (K),
5800 to 6000 mg/l of chloride (Cl),
20 to 400 mg/l of calcium (Ca),
50 to 1500 mg/l of magnesium (Mg).

According to a first advantageous embodiment, the seawater-based isoosmotic ionic solution used according to the invention has the following features:

pH of 7.8 to 8.4,

dry matter content of 1 to 2% by weight,

osmolarity of 305 to 315 mOsm/kg and the following chemical composition of the main elements,
for Na⁺, from 2000 to 2600,
for K⁺, from 40 to 80 mg/l,
for Mg⁺⁺, from 1200 to 1500 mg/l,
for Ca⁺⁺, from 300 to 400 mg/l,
for Cl⁻, from 5800 to 6000 mg/l.

According to a second advantageous embodiment, the seawater-based isoosmotic ionic solution used according to the invention has the following features:
pH of 7.0 to 9,
dry matter content of 1 to 2% by weight,
osmolarity of 250 to 350 mOsm/kg and the following chemical composition of the main elements,
for Na⁺, from 500 to 1500, preferentially from 1000 to 1300 mg/l,
for K⁺, from 4500 to 6500, preferentially from 5000 to 6000 mg/l,
for Mg⁺⁺, from 50 to 1300, preferentially from 100 to 500 mg/l,
for Ca⁺⁺, from 20 to 350, preferentially from 40 to 200 mg/l,
for Cl⁻, from 4000 to 6000, preferentially from 4500 to 5000 mg/l.

The seawater-based solution such as that used according to invention may be sterile or be sterilised.

Advantageously, the seawater-based solution also contains other elements such as bromine (Br), preferentially at least 50 mg/l, aluminium (Al), fluorine (F), iodine (I), iron (Fe), zinc (Zn), copper (Cu), manganese (Mn), selenium (Se).

Very advantageously, the composition consisting of elements other than sodium, potassium, chlorides, calcium and magnesium is qualitatively and quantitatively identical to that of seawater.

From a qualitative point of view, the ionic composition of the solutions used according to the invention is that of seawater.

As an illustration, table A below shows the composition of seawater as given on page F 163 of the publication “Handbook of Chemistry and Physics” 63rd edition, 1982-1983, CRC PRESS.

TABLE A

| Element | Quantity (ppm) |
|---|---|
| Cl | 18,980 |
| Na | 10,561 |
| Mg | 1,272 |
| S | 884 |
| Ca | 400 |
| K | 380 |
| Br | 65 |
| C (inorganic) | 28 |
| Sr | 13 |
| (SiO₂) | 0.01-7.0 |
| B | 4.6 |
| Si | 0.02-4.0 |
| C (organic) | 1.2-3.0 |
| Al | 0.16-1.9 |
| F | 1.4 |
| N (nitrate) | 0.001-0.7 |
| N (organic nitrogen) | 0.03-0.2 |
| Rb | 0.2 |
| Li | 0.1 |
| P (phosphate) | >0.001-0.10 |
| Ba | 0.05 |
| I | 0.05 |
| N (nitrite) | 0.0001-0.05 |
| N (ammoniac) | >0.005-0.05 |
| As (arsenic) | 0.003-0.024 |
| Fe | 0.002-0.02 |
| P (organic phosphorus) | 0.016 |
| Zn | 0.005-0.014 |
| Cu | 0.001-0.09 |
| Mn | 0.001-0.01 |
| Pb | 0.004-0.005 |
| Se | 0.004 |
| Sn | 0.003 |
| Cs | 0.002 (approximately) |
| U | 0.00015-0.0016 |
| Mo | 0.0003-0.002 |
| Ga | 0.0005 |
| Ni | 0.0001-0.0005 |
| Th | <0.0005 |
| Ce | 0.0004 |
| V | 0.0003 |
| La | 0.0003 |
| Y | 0.0003 |
| Hg | 0.00003 |
| Ag | 0.00015-0.0003 |
| Bi | 0.0002 |
| Co | 0.0001 |
| Sc | 0.00004 |
| Au | 0.000004-0.000008 |
| Fe (in true solution) | <10⁻⁹ |
| Ra | 2.10⁻¹¹-3.10⁻¹⁰ |
| Ge | Present |
| Ti | Present |
| W | Present |
| Cd | Present in marine organisms |
| Cr | Present in marine organisms |
| Tl | Present in marine organisms |
| Sb | Present in marine organisms |
| Zr | Present in marine organisms |
| Pt | Present in marine organisms |

On the same page of this publication, it is specified that the pH of seawater is 8-9.

It is also known (IFREMER, Coastal Environment and Marine Environment Management Department) that the osmolality of seawater is greater than 1000 mOsm/kg.

It is likewise known that, in raw seawater, the Na:Mg ratio is greater than 8.

In the case of seawater, the corresponding values are represented by ranges reflecting the results of 134 measurements made on seawater sampled off Saint-Malo from August 1998 to July 1999, i.e.:
pH: 7.70 to 8.30
osmolality: >1000 mOsm/kg
[Na⁺]: 10500-11500 mg/l
[K⁺]: 365-420 mg/l
[Mg⁺⁺]: 1200-1450 mg/l
[Ca⁺⁺]: 380-435 mg/l
[Cl⁻]: 18900-20500 mg/l

Na:Mg ratio: >8.

The ionic solutions used according to the invention are devoid of any preservatives or stabilisers. This advantage is of major importance.

Indeed, the preservatives and/or stabilisers present in the majority of synthetic ionic solutions, induce side effects more or less in the long-term. However, according to the invention, the ionic solution is administered over periods of time ranging from two weeks to several months, or even years.

The invention also relates to the use of a seawater-based isoosmotic ionic solution for preparing a medicinal product for administration by means of a nasal spray or mist, to patients suffering from the common cold or flu syndrome, or in the remission phase of the common cold or flu syndrome, to prevent and/or treat complications of the common cold of flu syndrome.

Said medicinal product may contain pharmaceutically acceptable excipients in addition to the seawater-based ionic solution. Preferentially, the medicinal product is devoid of preservatives or stabilisers.

According to one advantageous embodiment, in patients in the remission phase of the common cold or flu syndrome, the solution is administered daily, at least once a day and more preferentially three times a day outside any episode of common cold or flu syndrome, for at least one week, preferentially for at least two weeks.

According to another advantageous embodiment, the solution is administered to patients suffering from the common cold or flu syndrome, at a rate of 2 to 9, preferentially from 3 to 8, and more preferentially from 4 to 6 daily applications throughout the cold or flu syndrome episode and, in the remission phase, at a rate of 1 to 3 daily sprays for at least two weeks, it being understood that the dose is reduced following remission.

Given that the isoosmotic ionic solution used according to the invention does not display any side effects, liable to be associated with the presence of stabilisers in particular, said solution may be used daily all year round, preferentially throughout the epidemiological period.

The invention also relates to the use as described above whereby said solution is administered during the common cold or flu syndrome, without administering any other medicinal product either during the curative phase or during the preventive phase, i.e. not during the treatment of the common cold or flu syndrome, or in the following weeks. In this way, patients in remission from the common cold or flu syndrome, or patients suffering from the common cold or flu syndrome, may be treated without administering any of the agents selected in the group comprising an antibiotic, an antipyretic, a mucolytic, an antiinflammatory, an antiinfective, a nasal decongestant, a cough medicine and mixtures thereof.

Surprisingly, it was found that the isoosmotic ionic solutions according to the invention were tolerated very well by patients who did not complain of burning or stinging during administration, particularly using a spray. In addition, the tolerance to the product increases over time.

According to a particularly advantageous administration schedule, the solution is administered daily throughout the year, at a rate of at least one daily spray or mist, and a rate of at least two, preferentially at least three, and more preferentially at least six, daily sprays or mists during epidemiological episodes of the common cold or flu syndrome.

Particularly advantageously, the isoosmotic ionic solutions used according to the invention may be prepared by means of seawater electrodialysis. More specifically, in succession:
as the raw material, seawater with a salt content greater than or equal to 32 g/l is sampled, advantageously at a depth of 5 to 10 metres in a zone with strong current movements,
said water is analysed and settled,
sodium is removed from the settled water by means of electrodialysis until an osmolality between 250 and 350 mOsm/kg, preferentially between 305 and 315 mOsm/kg, is obtained,
the ionic concentrations are adjusted by means of selective electrodialysis,
the product is filtered and optionally stored under sterile conditions.

The use of this method makes it possible to adjust the concentrations of the main ionic species while retaining the quantitative and qualitative composition of all the other species found in seawater.

### Working Examples

In this example, an isoosmotic ionic solution marketed by GOËMAR under the brand PHYSIOMER® which consists of 100% undiluted sodium-free, sterile and preservative-free natural seawater and another isoosmotic ionic solution consisting of 100% sterile potassium-enriched natural seawater, marketed by GOËMAR under the brand SEROPHTA® are used.

The PHYSIOMER® solution used in this example is contained in a bottle fitted with a spray tube (PHYSIOMER® Spray) or in a bottle fitted with a jet tube (PHYSIOMER® Normal jet).

The SEROPHTA® solution is contained in a bottle fitted with a spray tube (SEROPHTA® Spray).

The efficacy of these isoosmotic ionic solutions was verified by a study conducted on a randomised population of 390 children of 6 to 10 years of age for 12 consecutive weeks.

The patients suffered from the common cold or flu syndrome at the start of the study.

The patients were divided into 4 homogeneous groups subjected to the following treatments, respectively:

group 1: Physiomer® Normal jet,

group 2: Physiomer® Spray,

group 3: Serophta® Spray,

group 4: control group, without nasal washing.

For groups 1 to 3, during weeks 0 to 3, the ionic solution was administered six times daily and for the subsequent weeks three times daily.

The patients of the four groups were observed by a doctor on the day of the start of the study (visit 1), one to three weeks (visit 2) and 6 to 8 weeks (visit 3) and 12 weeks (visit 4) after the start of the study.

Visit 1: diagnosis, enrolment in study, severity of nasal symptoms, administration of medicinal products;

Visit 2: examination of state of health, severity of nasal symptoms, optionally change of medicinal products, evaluation of efficacy by doctor and by patient, safety;

Visit 3: medical status-recurrence, evaluation of efficacy by patient, safety;

Visit 4: final examination of state of health, severity of nasal symptoms, evaluation of efficacy by doctor and by patient, safety.

The qualitative dry cough evaluation was transposed to a numeric scale as follows:

| Dry cough | None | Slight | Moderate | Severe |
|---|---|---|---|---|
| Scale | 1 | 2 | 3 | 4 |

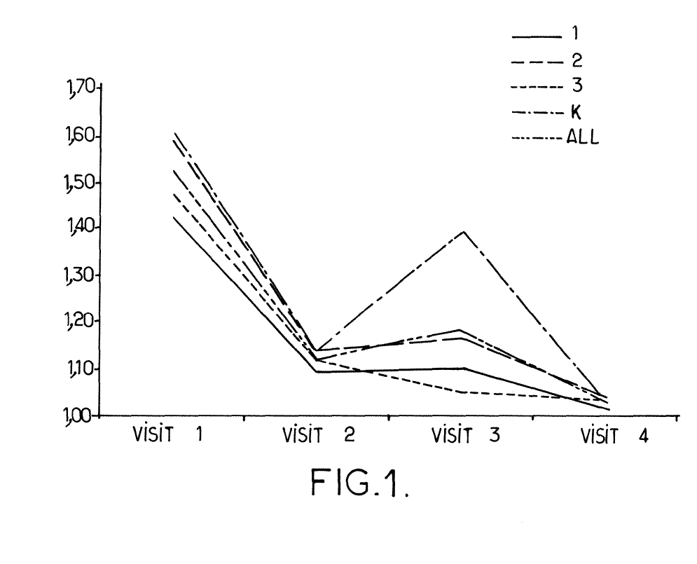

The results obtained are given in table 1 hereinafter and reproduced in graph form in FIG. 1:

TABLE 1

| | Group 1 | Group 2 | Group 3 | Group 4 |
|---|---|---|---|---|
| Visit 1 | 1.42 | 1.59 | 1.47 | 1.60 |
| Visit 2 | 1.09 | 1.14 | 1.12 | 1.14 |
| Visit 3 | 1.10 | 1.17 | 1.05 | 1.40 |
| Visit 4 | 1.02 | 1.04 | 1.03 | 1.04 |

A noteworthy difference appears at visit 3 between the patients of groups 1, 2 or 3 and those of group 4 (control).

The quantitative nasal secretion evaluation was transposed to a numeric scale as follows:

| Nasal secretion | None | Low | Moderate | Severe |
|---|---|---|---|---|
| Scale | 1 | 2 | 3 | 4 |

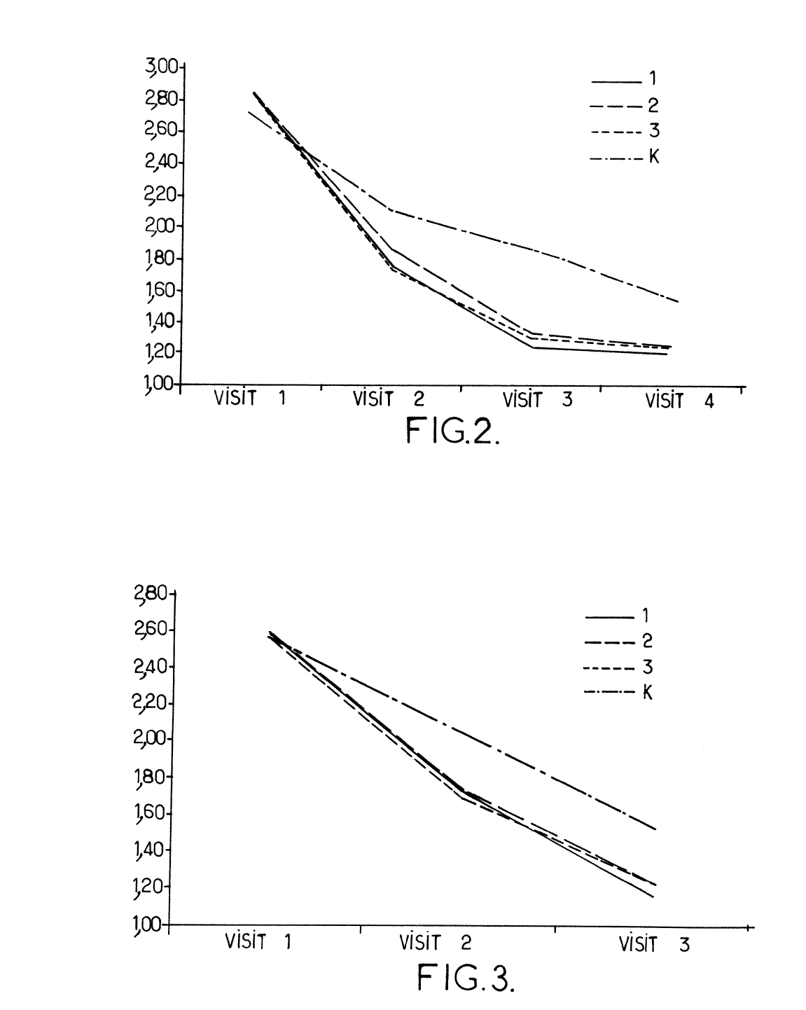

The results obtained are given in table 2 hereinafter and reproduced in graph form in FIG. 2:

TABLE 2

| | Group 1 | Group 2 | Group 3 | Group 4 |
|---|---|---|---|---|
| Visit 1 | 2.58 | 2.84 | 2.83 | 2.70 |
| Visit 2 | 1.76 | 1.86 | 1.74 | 2.10 |
| Visit 3 | 1.24 | 1.33 | 1.30 | 1.86 |
| Visit 4 | 1.20 | 1.24 | 1.23 | 1.55 |

The difference between the patients of groups 1, 2 or 3 and those of group 4 (control) was significant at visits 2, 3 and 4.

The qualitative nasal secretion evaluation was transposed to a numeric scale as follows:

Nasal secretion

| | None | Seric | Seropurulent | Purulent |
|---|---|---|---|---|
| Scale | 1 | 2 | 3 | 4 |

The results obtained are given in table 3 hereinafter and reproduced in graph form in FIG. 3:

TABLE 3

| | Group 1 | Group 2 | Group 3 | Group 4 |
|---|---|---|---|---|
| Visit 1 | 2.59 | 2.58 | 2.56 | 2.55 |
| Visit 2 | 1.74 | 1.74 | 1.69 | 2.06 |
| Visit 4 | 1.16 | 1.23 | 1.23 | 1.53 |

For visits 2 and 4, there is a significant difference between the nasal secretions of groups 1, 2 or 3 with respect to group 4.

The ability to breathe through the nose was transposed to a numeric scale as follows:

Breathing through the nose

| | | Slight | Moderate | |
|---|---|---|---|---|
| | No difficulty | difficulty | difficulty | Impossible |
| Scale | 1 | 2 | 3 | 4 |

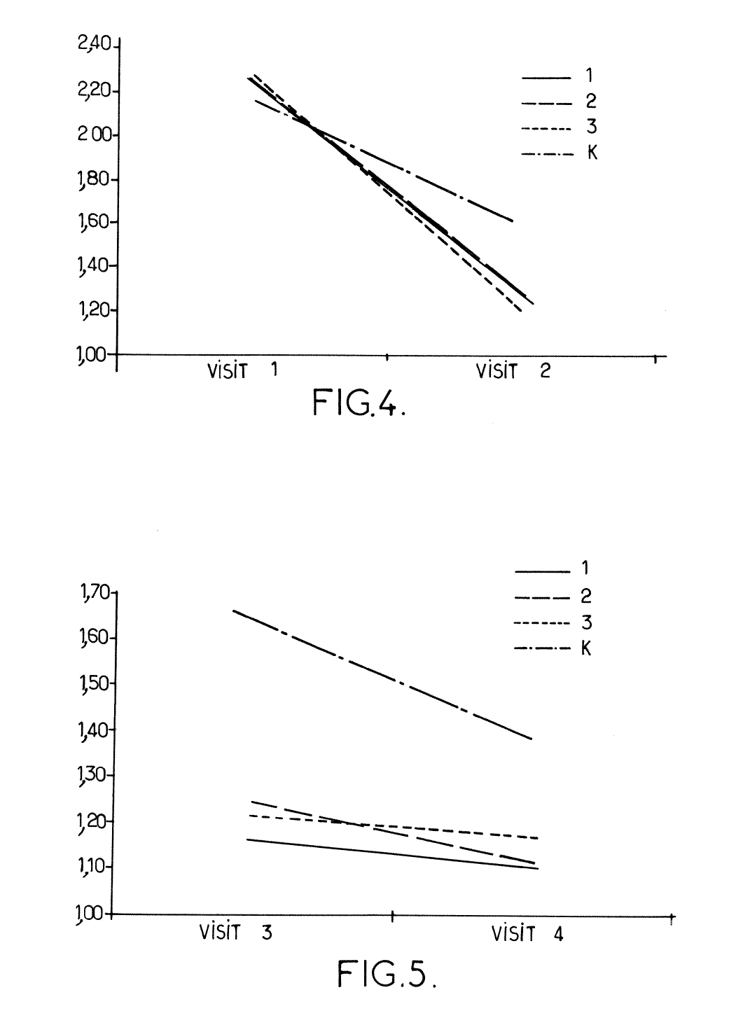

The results obtained are given in table 4 hereinafter and reproduced in graph form in FIG. 4:

TABLE 4

| | Group 1 | Group 2 | Group 3 | Group 4 |
|---|---|---|---|---|
| Visit 1 | 2.26 | 2.24 | 2.27 | 2.16 |
| Visit 2 | 1.27 | 1.28 | 1.20 | 1.58 |

The patients of groups 1, 2 and 3 experienced a significant improvement in breathing through the nose, whereas the patients of group 4 only experienced a slight improvement.

Nasal obstruction was transposed to a numeric scale as follows:

Nasal obstruction

| | None | Slight | Moderate | Severe |
|---|---|---|---|---|
| Scale | 1 | 2 | 3 | 4 |

The results obtained are given in table 5 hereinafter and reproduced in graph form in FIG. 5:

TABLE 5

| | Group 1 | Group 2 | Group 3 | Group 4 |
|---|---|---|---|---|
| Visit 3 | 1.16 | 1.24 | 1.21 | 1.64 |
| Visit 4 | 1.10 | 1.12 | 1.17 | 1.39 |

At visit 3 and visit 4, the nasal obstruction in the patients of group 4 (control) was greater than that of the patients of groups 1, 2 or 3.

The administration of medicinal products was specified, particularly antipyretics, antiinfectives, nasal decongestants and mucolytics.

The percentage of patients from each group to whom antipyretics were administered was specified at each visit.

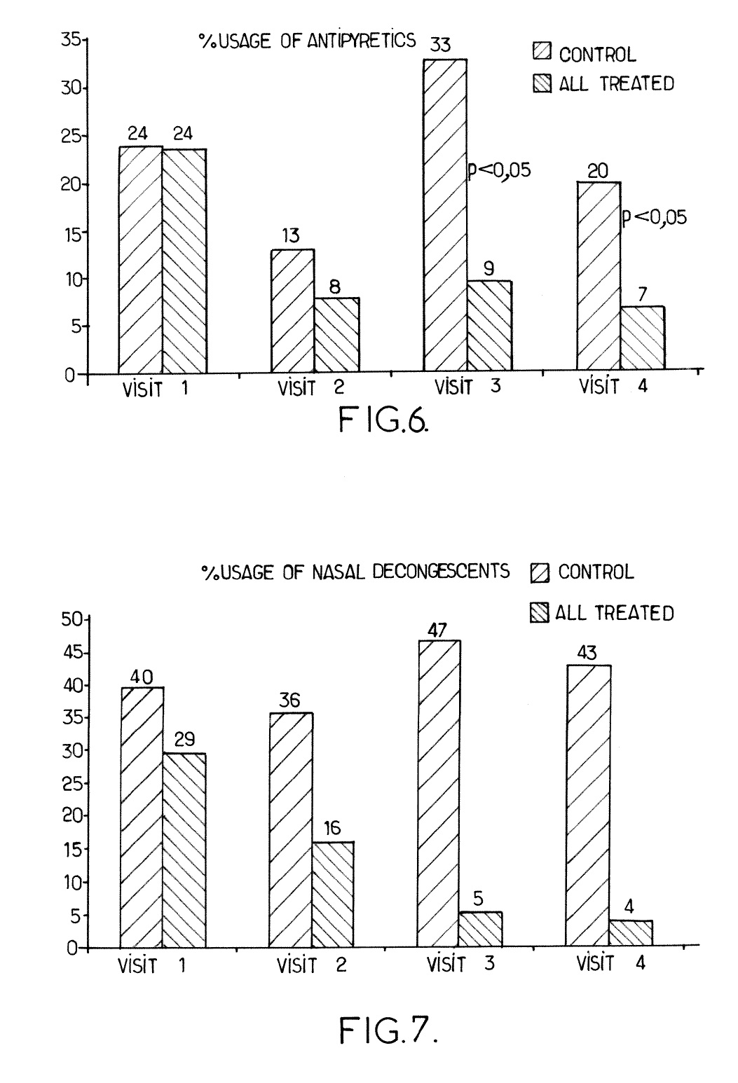

These results are given in table 6 and in FIG. 6.

TABLE 6

| | Group 1 | Group 2 | Group 3 | Group 4 |
|---|---|---|---|---|
| Visit 1 | 26 | 23 | 21 | 24 |
| Visit 2 | 5 | 7 | 11 | 13 |
| Visit 3 | 9 | 8 | 11 | 33 |
| Visit 4 | 8 | 5 | 6 | 20 |

The number of patients from group 4 (control) taking antipyretics at visits 3 and 4 is markedly greater than that of groups 1, 2 or 3.

The percentage of patients from each group to whom nasal decongestants were administered was specified at each visit.

These results are given in table 7 and in FIG. 7.

TABLE 7

| | Group 1 | Group 2 | Group 3 | Group 4 |
|---|---|---|---|---|
| Visit 1 | 29 | 27 | 32 | 40 |
| Visit 2 | 15 | 14 | 19 | 36 |
| Visit 3 | 6 | 6 | 3 | 47 |
| Visit 4 | 4 | 7 | 0 | 43 |

The number of patients from group 4 (control) taking nasal decongestants at visit 4 is markedly greater than that of groups 1, 2 or 3.

The percentage of patients from each group to whom antiinfectives were administered was specified at each visit.

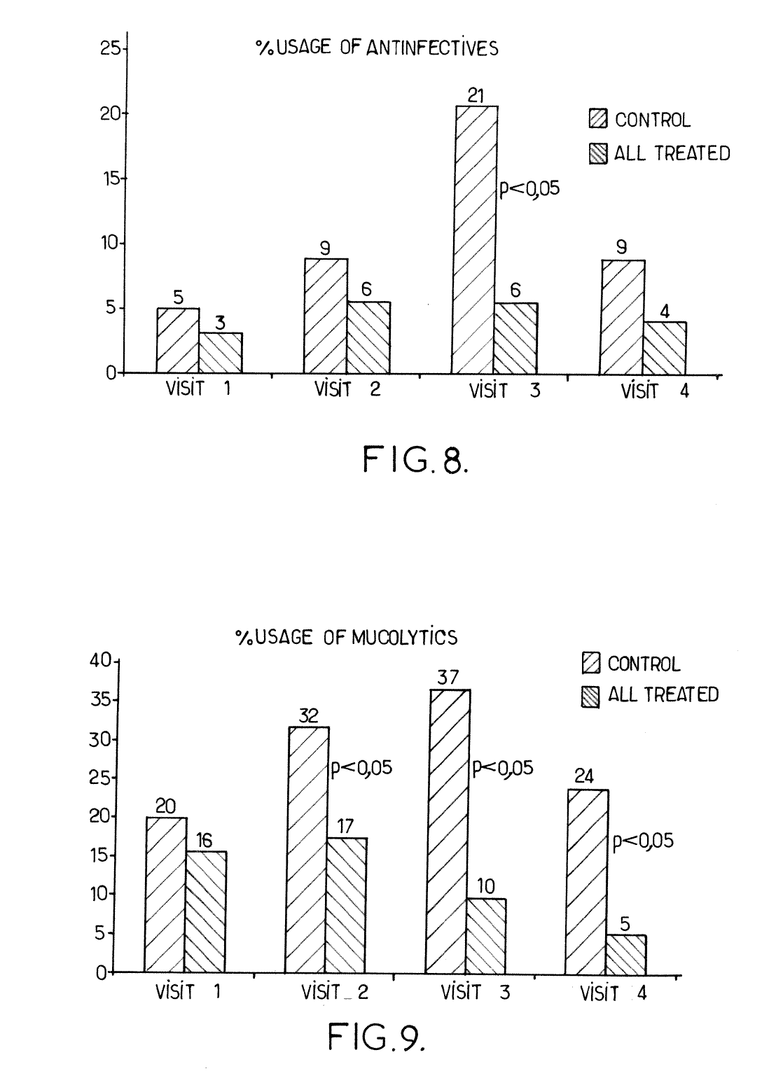

These results are given in table 8 and in FIG. 8.

TABLE 8

| | Group 1 | Group 2 | Group 3 | Group 4 |
|---|---|---|---|---|
| Visit 1 | 6 | 1 | 2 | 5 |
| Visit 2 | 7 | 6 | 3 | 9 |
| Visit 3 | 4 | 5 | 7 | 21 |
| Visit 4 | 6 | 3 | 3 | 9 |

The percentage of systemic antiinfective administration was very low in each group. A statistically significant difference between the patients of groups 1, 2 or 3 and those of group 4 (control) was only observed for visit 3.

The percentage of patients from each group to whom mucolytics were administered was specified at each visit.

These results are given in table 9 and in FIG. 9.

TABLE 9

| | Group 1 | Group 2 | Group 3 | Group 4 |
|---|---|---|---|---|
| Visit 1 | 15 | 14 | 18 | 20 |
| Visit 2 | 15 | 15 | 22 | 32 |
| Visit 3 | 9 | 9 | 11 | 37 |
| Visit 4 | 4 | 4 | 6 | 24 |

From visit 2, noteworthy differences appear between the patients of groups 1, 2 or 3 and those of group 4.

The recurrence of the disease was measured by determining the percentage of patients reporting sick days during the prevention phase, i.e. between weeks 4 and 12 and by counting the number of days when children did not attend school due to their state of health.

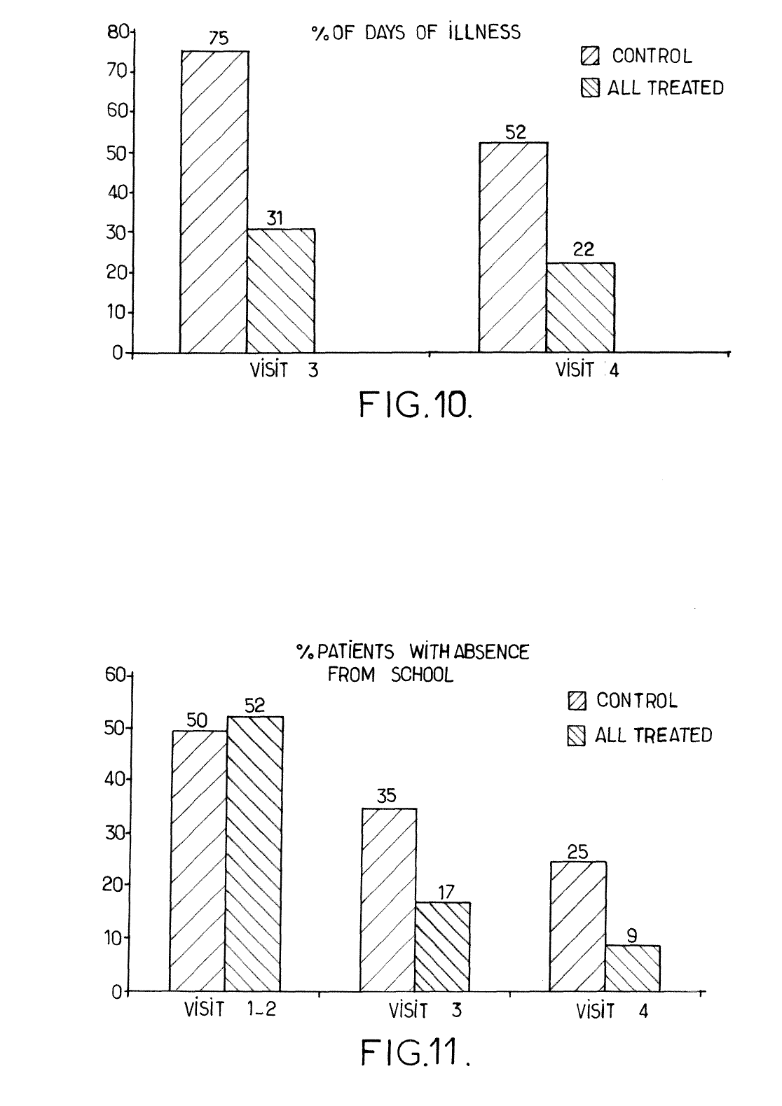

The percentage of children who had been ill since the previous visit is given in table 10 and the results are recorded in FIG. 10:

TABLE 10

| | Group 1 | Group 2 | Group 3 | Group 4 |
|---|---|---|---|---|
| Visit 3 | 27 | 35 | 31 | 75 |
| Visit 4 | 22 | 22 | 22 | 52 |

Between visit 2 and visit 3, 75% of the patients of group were ill, i.e. approximately 60% more than among the patients of groups 1, 2 or 3.

The percentage of children who had missed at least one day since the previous visit is given in table 11 and the results are recorded in FIG. 11:

TABLE 11

| | Group 1 | Group 2 | Group 3 | Group 4 |
|---|---|---|---|---|
| Visits 1-2 | 53 | 52 | 53 | 50 |
| Visit 3 | 16 | 14 | 21 | 35 |
| Visit 4 | 7 | 8 | 11 | 25 |

A 50 to 60% decrease in the days missed is observed between the patients of groups 1, 2 or 3 and those of group 4.

The general state of health of the children at the start of the study and at the end thereof was evaluated by the parents and was transposed to a numeric scale as follows:

General state of health

| | | | | Not |
|---|---|---|---|---|
| | Excellent | Good | Satisfactory | satisfactory |
| Scale | 1 | 2 | 3 | 4 |

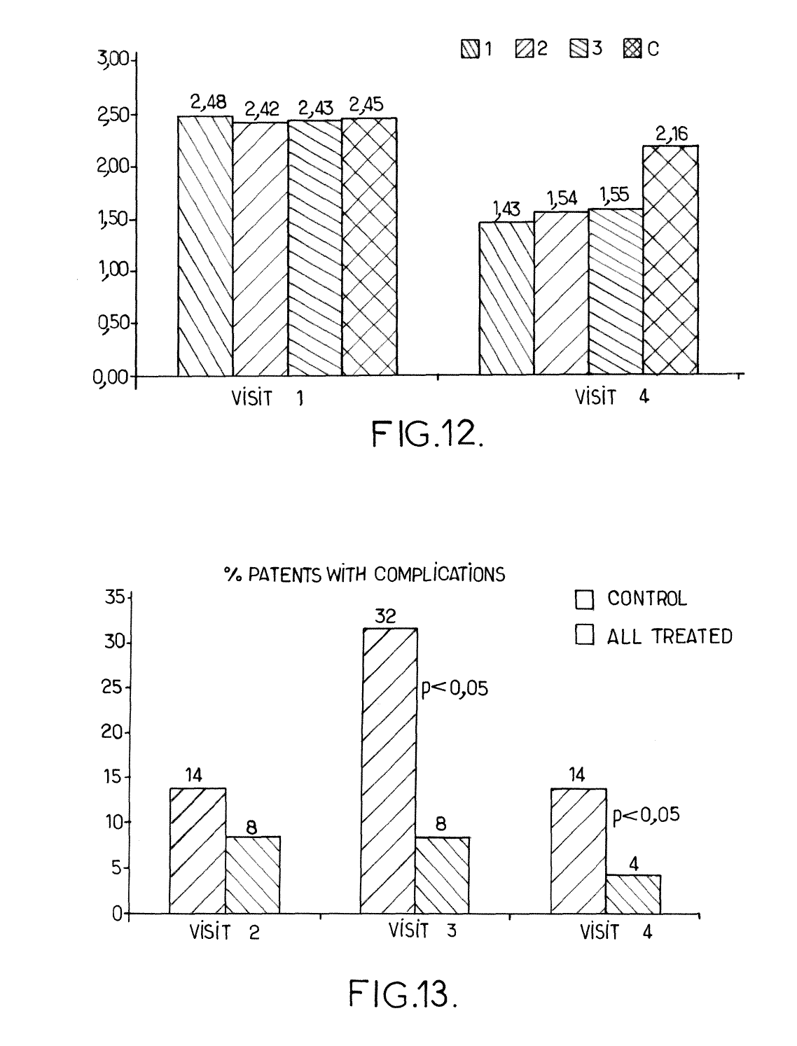

The results obtained are given in table 12 hereinafter and reproduced in graph form in FIG. 12:

TABLE 12

| | Group 1 | Group 2 | Group 3 | Group 4 |
|---|---|---|---|---|
| Visit 1 | 2.48 | 2.42 | 2.43 | 2.45 |
| Visit 4 | 1.43 | 1.54 | 1.55 | 2.16 |

At the end of the study, the general state of health of the patients of groups 1, 2 or 3 was significantly superior to that of the general state of health of the patients of group 4.

The percentage of patients displaying complications during the study is given in table 13 hereinafter and reproduced in graph form in FIG. 13:

TABLE 13

| | Group 1 | Group 2 | Group 3 | Group 4 |
|---|---|---|---|---|
| Visit 2 | 10 | 4 | 11 | 14 |
| Visit 3 | 8 | 7 | 10 | 32 |
| Visit 4 | 6 | 3 | 3 | 14 |

In view of these results, it would appear that the isoosmotic solutions according to the invention represent an effective solution to preventing the complications of the common cold. They enable a quicker resolution of nasal symptoms, lower medicinal product consumption, a quicker improvement in the general state of health and also make it possible to limit the number of schooldays missed.

What is claimed is:

1. A method of treating a patient, comprising: administering a seawater-based isoosmotic ionic solution by nasal spray or mist to patients in the remission phase of the common cold or flu syndrome, or to patients suffering from the common cold or flu syndrome, to prevent and/or treat complications of the common cold or flu syndrome.

2. The method according to claim 1, wherein the seawater-based isoosmotic ionic solution comprises:
a pH of 7.8 to 8.4,
a dry matter content of 1 to 2% by weight,
an osmolarity of 250 to 350 mOsm/kg, preferentially 305 to 315 mOsm/kg, and the following main constituent contents:
500 to 2600 mg/l of sodium (Na),
40 to 6500 mg/l of potassium (K),
5800 to 6000 mg/l of chloride (Cl),
20 to 400 mg/l of calcium (Ca), and
50 to 1500 mg/l of magnesium (Mg).

3. The method according to claim 1, wherein the seawater-based isoosmotic ionic solution comprises:
pH of 7.8 to 8.4,
dry matter content of 1 to 2% by weight,
osmolarity of 305 to 315 mOsm/kg and the following chemical composition of the main elements,
for Na⁺, from 2000 to 2600,
for K⁺, from 40 to 80 mg/l,
for Mg⁺⁺, from 1200 to 1500 mg/l,
for Ca⁺⁺, from 300 to 400 mg/l, and
for Cl⁻, from 5800 to 6000 mg/l.

4. The method according to claim 1, wherein the seawater-based isoosmotic ionic solution comprises:
pH of 7.0 to 9,
dry matter content of 1 to 2% by weight,
osmolarity of 250 to 350 mOsm/kg and the following chemical composition of the main elements,
for Na⁺, from 500 to 1500, preferentially from 1000 to 1300 mg/l,
for K⁺, from 4500 to 6500, preferentially from 5000 to 6000 mg/l,
for Mg⁺⁺, from 50 to 1300, preferentially from 100 to 500 mg/l,
for Ca⁺⁺, from 20 to 350, preferentially from 40 to 200 mg/l, and
for Cl⁻, from 4000 to 6000, preferentially from 4500 to 5000 mg/l.

5. The method according to claim 1, wherein the seawater-based isoosmotic ionic solution also contains bromine (Br), preferentially at least 50 mg/l, aluminium (Al), fluorine (F), iodine (I), iron (Fe), zinc (Zn), copper (Cu), manganese (Mn), and/or selenium (Se).

6. The method according to claim 4, wherein, in the seawater-based isoosmotic ionic solution, the composition consisting of elements other than sodium, potassium, chlorides, calcium and magnesium is qualitatively and quantitatively identical to that of seawater.

7. The method according to claim 1, wherein the solution is devoid of any preservatives or stabilisers.

8. The method according to claim 1, wherein the solution is administered daily by nasal spray or mist, at least once a day and preferentially at least three times a day in each nostril for at least one week, preferentially for at least two weeks.

9. The method according to a claim 1, wherein the solution is administered to patients suffering from the common cold or flu syndrome, at a rate of 2 to 9, preferentially from 3 to 8, and more preferentially from 4 to 6 daily applications throughout the cold or flu syndrome episode and, in the remission phase, at a rate of 1 to 3 daily sprays for at least two weeks, wherein the dose is reduced following remission.

10. The method according to claim 1, wherein the solution is administered all year round, preferentially throughout the epidemiological period.

11. The method according to claim 1, wherein the solution is administered to patients suffering from the common cold or flu syndrome, or in the remission phase of the common cold or flu syndrome, without administering any of the agents selected in the group comprising an antibiotic, an antipyretic, a mucolytic, an antiinflammatory, an antiinfective, a nasal decongestant, a cough medicine and mixtures thereof, during the curative phase and/or preventive phase.
